(19) United States
(12) Patent Application Publication
(10) Pub. No.: US 2009/0171284 A1
(43) Pub. Date: Jul. 2, 2009

(54) DILATION SYSTEM

(75) Inventors: Jessica Louise Burke, Bloomington, IN (US); Jeffry S. Melsheimer, Springville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/965,484

(22) Filed: Dec. 27, 2007

(51) Int. Cl. A61M 29/00

(52) U.S. Cl. 604/104

(57) ABSTRACT

A dilation system and method of use thereof are provided that may be used to dilate hardened regions of a stenosis. The dilation system is provided with dilation elements that extend between a catheter and a distal tip to form a cage-like structure. The inner passageway of the cage-like structure is sized to receive a balloon catheter. During a procedure, the balloon catheter may be introduced into the cage. Inflation of the balloon causes the dilation elements to radially move outward and contact a stenosed region. After dilation of the stenosed region, the balloon catheter may be withdrawn.

### BACKGROUND

The present invention relates generally to medical devices and more particularly to catheters used to dilate narrowed portions of a lumen.

Balloon catheters are widely used in the medical profession for various intraluminal procedures. One common procedure involving the use of a balloon catheter relates to angioplasty dilation of coronary or other arteries suffering from stenosis (i.e., a narrowing of the arterial lumen that restricts blood flow).

Although balloon catheters are used in many other procedures as well, coronary angioplasty using a balloon catheter has drawn particular attention from the medical community because of the growing number of people suffering from heart problems associated with stenosis. This has lead to an increased demand for medical procedures to treat such problems. The widespread frequency of heart problems may be due to a number of societal changes, including the tendency of people to exercise less while eating greater quantities of unhealthy foods, in conjunction with the fact that people generally now have longer life spans than previous generations. Angioplasty procedures have become a popular alternative for treating coronary stenosis because angioplasty procedures are considerably less invasive than other alternatives. For example, stenosis of the coronary arteries has traditionally been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

To address the increased need for coronary artery treatments, the medical community has turned to angioplasty procedures, in combination with stenting procedures, to avoid the problems associated with traditional bypass surgery. Typically, angioplasty procedures are performed using a balloon-tipped catheter that may or may not have a stent mounted on the balloon (also referred to as a stented catheter). The physician performs the angioplasty procedure by introducing the balloon catheter into a peripheral artery (commonly one of the leg arteries) and threading the catheter to the narrowed part of the coronary artery to be treated. During this delivery stage, the balloon is uninflated and collapsed onto the shaft of the catheter in order to present a low profile which may be passed through the arterial lumens. Once the balloon is positioned at the narrowed part of the artery, the balloon is expanded by pumping a mixture of saline and contrast solution through the catheter to the balloon. As a result, the balloon presses against the inner wall of the artery to dilate it. If a stent is mounted on the balloon, the balloon inflation also serves to expand the stent and implant it within the artery. After the artery is dilated, the balloon is deflated so that it once again collapses onto the shaft of the catheter. The balloon-tipped catheter is then retracted from the arteries. If a stent is mounted on the balloon of the catheter, the stent is left permanently implanted in its expanded state at the desired location in the artery to provide a support structure that prevents the artery from collapsing back to its pre-dilated condition. On the other hand, if the balloon catheter is not adapted for delivery of a stent, either a balloon-expandable stent or a self-expandable stent may be implanted in the dilated region in a follow-up procedure. Although the treatment of stenosed coronary arteries is one common example where balloon catheters have been used, this is only one example of how balloon catheters may be used and many other uses are also possible.

One problem that may be encountered with conventional angioplasty techniques is the proper dilation of stenosed regions that are hardened and/or have become calcified. Stenosed regions may become hardened for a variety of reasons, such as the buildup of atherosclerotic plaque or other substances. Hardened regions of a stenosis can be difficult to completely dilate using conventional balloons because hardened regions tend to resist the expansion pressures applied by conventional balloon catheters. Although the inventions described below may be useful in treating hardened regions of stenoses, the claimed inventions may also solve other problems as well.

### SUMMARY

A dilation system is provided that may be used to dilate hardened regions of a stenosis. The dilation system is provided with dilation elements that extend between a catheter and distal tip to form a cage-like region therebetween. The inner passageway of the cage-like structure is sized to receive a balloon catheter. During a procedure, the balloon catheter may be introduced into the cage. Inflation of the balloon causes the dilation elements affixed between the catheter and distal tip to radially move outward and contact a stenosed region. After dilation of the stenosed region, the balloon catheter may be deflated and withdrawn. Additional details and advantages are described below in the detailed description.

The invention may include any of the following aspects in various combinations and may also include any other aspect described below in the written description or in the attached drawings.

A dilation system for dilation of a vessel wall, comprising: a catheter comprising a distal end and a proximal end; a distal tip distally spaced apart a predetermined distance from the catheter; a plurality of dilation elements extending between the catheter and the distal tip, the plurality of dilation elements defining a cage, and a balloon removably slidably disposed within the cage, the balloon mounted on the distal end of a shaft, the balloon having a distal portion, a proximal portion, wherein at least a length of an outer surface of the balloon comprises a working diameter adapted to dilate the vessel wall, the working diameter of the balloon longitudinally aligned and extending within the cage, the shaft having an inflation lumen extending therethrough in fluid communication with an interior region of the balloon, the balloon thereby being expandable between a deflated state and an inflated state.

The dilation system, wherein the catheter and the distal tip comprise multiple lumens configured to receive each of the plurality of dilation elements.

The dilation system, wherein each of the plurality of dilation elements is molded to the distal tip.

The dilation system, wherein each of the plurality of dilation elements are equally spatially apart and longitudinally aligned with respect to each other.

The dilation system, wherein each of the plurality of dilation elements is affixed by an adhesive.

The dilation system, wherein the cage is characterized by an inner passageway.

The dilation system, wherein the inner passageway comprises a longitudinal length that is at least about equal to a length of the working diameter of the balloon.

The dilation system, wherein the plurality of dilation elements are movable between a cage-like configuration and a radially bowed orientation.

The dilation system, wherein the plurality of dilation elements freely extend along the balloon.

The dilation system, wherein at least one end of the plurality of dilation elements is fastened to a collar crimped on at least one of the catheter and the distal tip

The dilation system, wherein the collar and/or plurality of dilation elements comprises a radiopaque indicia.

The dilation system, wherein each of the plurality of dilation elements comprises a non-circular cross section.

A dilation system for dilation of a vessel wall, comprising: a catheter comprising a distal end and a proximal end; a plurality of wires comprising a proximal end heat bonded to the distal end of the catheter and a distal end heat bonded to a distal tip, the plurality of wires defining a cage, and a balloon removably slidably disposed within the cage, the balloon mounted on the distal end of a shaft, the balloon having a distal portion, a proximal portion, wherein at least a length of an outer surface of the balloon comprises a working diameter adapted to dilate the vessel wall, the working diameter of the balloon longitudinally extending and aligned within the cage, the shaft having an inflation lumen extending therethrough in fluid communication with an interior region of the balloon, the balloon thereby being expandable between a deflated state and an inflated state.

The dilation system, wherein the catheter further comprises one or more heat bonded layers.

The dilation system, wherein each of the plurality of wires comprises a cross-sectional shape that is adapted to bidirectionally flex.

The dilation system, wherein the distal tip and catheter comprise multiple lumens to receive the plurality of wires.

A method of dilating a stenosis in a body vessel, comprising the steps of: (a) providing a catheter comprising a distal end and a proximal end; a distal tip distally spaced apart a predetermined distance from the catheter; a plurality of dilation elements extending between the catheter and the distal tip, the plurality of dilation elements defining a cage, and a balloon mounted on the distal end of a shaft, the balloon having a distal portion, a proximal portion, wherein at least a length of an outer surface of the balloon comprises a working diameter adapted to dilate the vessel wall, the shaft having an inflation lumen extending therethrough in fluid communication with an interior region of the balloon, the balloon thereby being expandable between a deflated state and an inflated state; (b) advancing the catheter to the target site; (c) advancing the cage of the first catheter to the target site; (d) advancing the expandable member of the second catheter to the target site until a first stopper element of the first catheter abuts against a second stopper element of the second catheter; and (e) expanding the expandable member, wherein each of the plurality of dilation elements expand with expandable member from a cage-like configuration to a radially expanded configuration toward a stenosed region.

The method, further comprising the steps of: (f) deflating the balloon; (g) returning the dilation elements from the radially outwards configuration to the cage-like configuration; and (h) withdrawing the balloon from the cage of the catheter.

The method of inflating the balloon comprises inflating the balloon to an inflation pressure between about 4 atm to about 9 atm.

### BRIEF DESCRIPTION OF SEVERAL VIEWS OF THE DRAWINGS

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

The invention may be more fully understood by reading the following description in conjunction with the drawings, in which:

FIG. 1 illustrates a dilation system comprising a balloon catheter and catheter, the catheter being spaced apart from a distal tip with dilation elements extending therebetween;

FIG. 2 shows a perspective view of the structure of FIG. 1 in which the balloon catheter is inserted into the cage and inflated therewithin to cause the dilation elements to radially move outwards;

FIG. 3 shows the dilation elements prior to being heat bonded to the multi-layered distal tip; and

FIG. 4 is a blown-up view of FIG. 3 showing a single dilation element in the process of being heat bonded to the multi-layered distal tip;

FIGS. 5A-5E show various shaped dilation elements disposed between an inner layer and outer layer; and

FIGS. 6-10 show a method of use of the dilation system within a stenosed vessel wall.

### DETAILED DESCRIPTION

The terms “dilation” and “dilating” as used herein denote the fracturing, cutting, and/or dilating of a stenosed region within a vessel wall.

The terms “distal” and “distally” shall denote a position, direction, or orientation that is generally away from the patient. Accordingly, the terms “proximal” and “proximally” shall denote a position, direction, or orientation that is generally towards the patient.

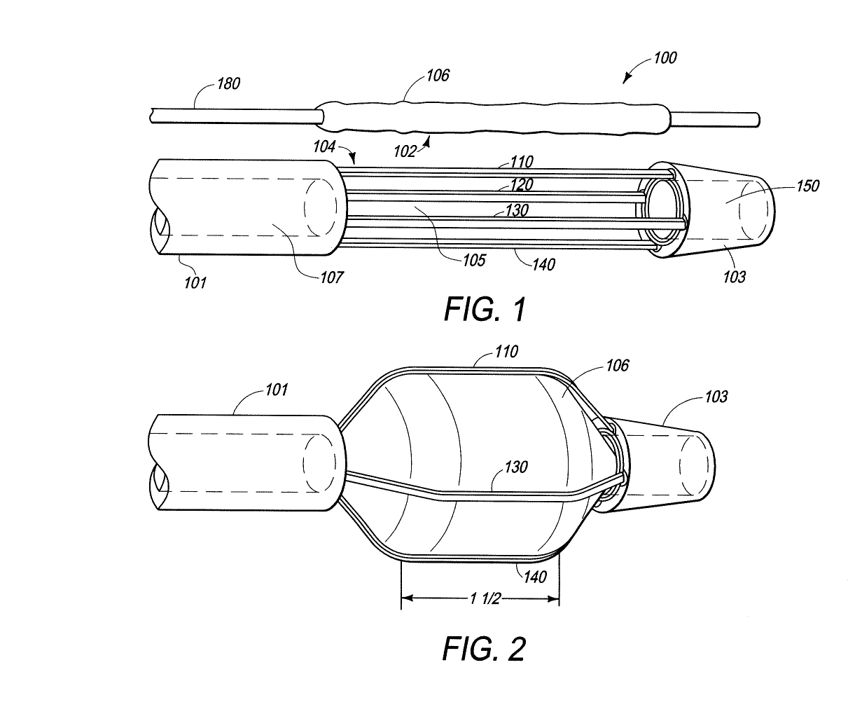

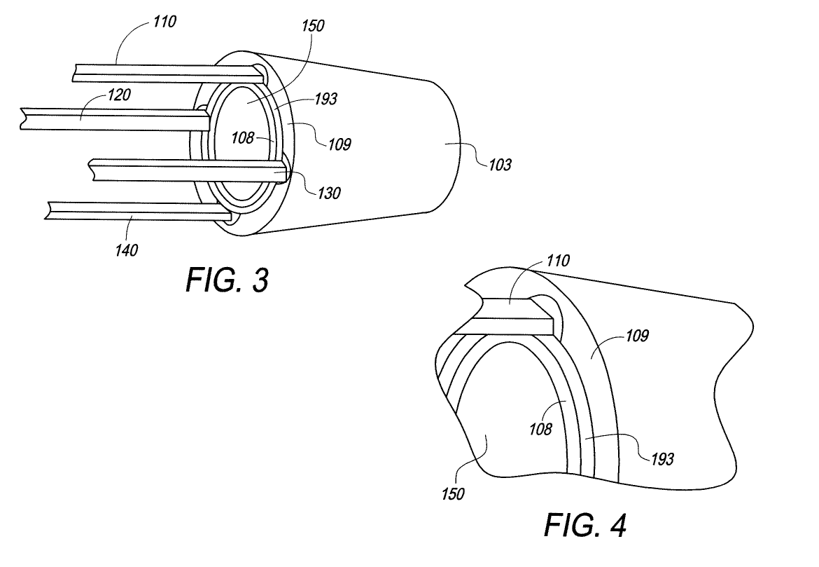

FIG. 1 illustrates an exemplary dilation system 100. The dilation system 100 comprises a catheter 101, a distal tip 103, dilation elements 110, 120, 130, 140 and a balloon catheter 102. Dilation elements 110, 120, 130, and 140 extend between the catheter 101 and the distal tip 103. The distal tip 103 may be distally spaced apart a predetermined distance from the catheter 101. The dilation elements 110, 120, 130, 140 may be spatially configured between the catheter 101 and the distal tip 103 to define a cage-like structure 104. The cage 104 may be characterized by an inner passageway 105 that is sized to allow a balloon catheter 102 to be introduced therein. The balloon catheter 102 is introduced through the working lumen 107 of the catheter 101 and into the passageway 105 of the catheter 101 such that the balloon 106 is disposed within the cage 104 and confined therewithin during a procedure. Preferably, the inner passageway 105 comprises a longitudinal length that is at least about equal to a length W_{L} of the working diameter of the balloon 106. Generally speaking, after the balloon catheter 102 is positioned within the cage 104, the balloon 106 may be inflated to its working diameter (FIG. 2). Because the proximal ends of the dilation elements 110, 120, 130, 140 are affixed to the end of the catheter 101 and the distal ends of the dilation elements 110, 120, 130, 140 are affixed to the distal tip 103, the inflation of the balloon 106 forces the unattached portions of the dilation elements 110, 120, 130, 140 to bow outwards (FIG. 2) and contact a stenosed vessel wall 600 (FIG. 10). Each of the dilation elements 110, 120, 130, 140 is sufficiently elastic to allow them to return from an expanded shape to a collapsed shape.

The forces resulting from inflating the balloon 106 are concentrated and focused along the dilation elements 110, 120, 130, 140 to dilate the stenosed vessel wall 600. The dilation mechanism may involve dilation or fracturing of the stenosed vessel wall 600. The dilation technique may also minimize the vascular trauma typically incurred during conventional balloon angioplasty because a lower balloon pressure can be applied compared to conventional angioplasty balloons. Typically, the working diameter of the balloon 106 (FIG. 2) is a portion that inflates to a generally uniform circumference in order to evenly dilate a section of a lumen. However, if desired, the working diameter does not necessarily need to have a uniform circumference.

As FIG. 1 shows, the dilation elements 110, 120, 130, 140 are preferably longitudinally aligned with respect to each other and oriented circumferentially about 90° relative to each other in their relaxed state. The dilation elements are movable between their natural, relaxed configuration shown in FIG. 1 and a radially bowed configuration shown in FIG. 2. In particular, the example of FIG. 2 shows that inflation of the balloon 106 forces the unattached portions of the dilation elements 110, 120, 130, 140 to bow outwards and yet remain longitudinally aligned with respect to each other in the bowed configuration. Specifically, FIG. 2 shows that the dilation elements 110, 120, 130, 140 in their radial bowed configuration remain oriented circumferentially about 90° relative to each other in their relaxed state. Other configurations, such as a helical configuration, are contemplated and would be appreciated by one of ordinary skill in the art.

In the example shown in FIG. 1, the dilation elements 110, 120, 130, 140 have a rectangular cross-sectional area. The dilation elements 110, 120, 130, 140 may be bonded to the surface of the catheter 101 and the surface of the distal tip 103. The dilation elements 110, 120, 130, 140 are preferably heat bonded, as will be explained in greater detail below. The sections of the dilation elements 110, 120, 130, 140 between the heat bonded regions are shown as unattached to define the cage 104 region. The cage 104 may have a length and width that is sufficient for the balloon catheter 102 to be introduced from the proximal end of the catheter 101 into the working lumen 107. The cage 104 may have a length from about 2.5 mm to about 40 mm and a width from about 1.0 mm to about 2.4 mm. In the example of FIG. 1, the cage 104 is sized to accommodate a balloon ranging in size from about 1 Fr to about 10 Fr, and more preferably sized to accommodate a balloon ranging in size from about 3 Fr to about 7 Fr. The balloon 106 is preferably disposed within the cage 104 of dilation elements 110, 120, 130, 140 (FIG. 7).

The balloon catheter 102 may be a typical angioplasty balloon catheter as used in the art. The balloon 106 is mounted on the distal end of a shaft 180 and comprises a distal portion and a proximal portion. At least a length of an outer surface of the balloon 106 comprises a working diameter in which the working diameter longitudinally extends within the cage 104. The shaft 180 comprises an inflation lumen extending therethrough which is in fluid communication with an interior region of the balloon 106. The inflation lumen causes the balloon 106 to be expandable between a deflated state and an inflated state.

The dilation elements 110, 120, 130, 140 may possess sufficient elasticity and/or flexibility such that the elements 110, 120, 130, 140 are movable in the radially outward direction while maintaining the circumferential orientation of the elements 110, 120, 130, 140 during movement in the radial outward direction. To accomplish this restricted movement in substantially only the radial direction, the dilation elements 110, 120, 130, and 140 may have a width-to-thickness ratio greater than 1, in which the thickness is defined in the radial direction and the width is defined in the circumferential direction. FIG. 1 shows the rectangular dilation elements 110, 120, 130, and 140 having a width-to-thickness ratio greater than 1. The relatively smaller thickness dimension may allow radial movement, and the relatively larger width dimension may prevent substantial lateral movement and rotational movement.

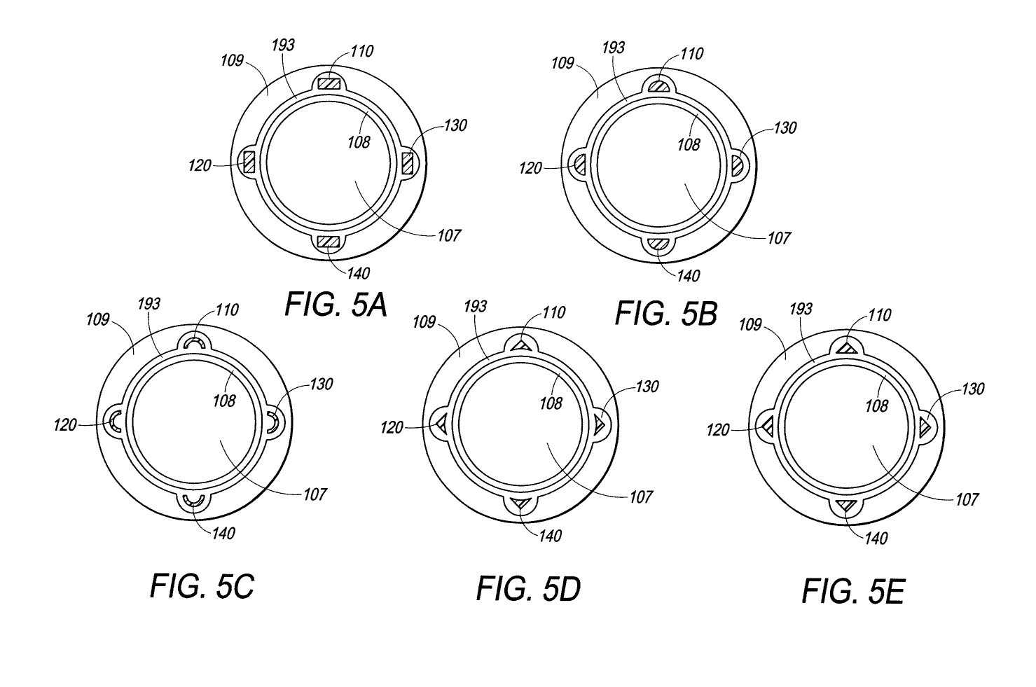

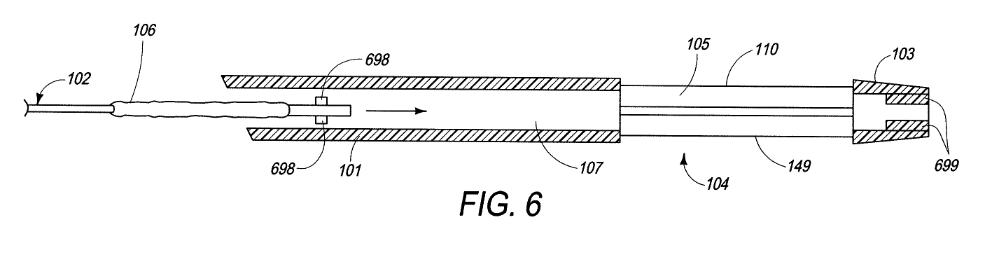

The dilation elements 110, 120, 130, 140 may be affixed to the distal tip 103 and the catheter 101 by any means known to one of ordinary skill in the art. In the example of FIGS. 3 and 4, the distal end of each of the dilation elements 110, 120, 130, 140 is shown in the process of being heat bonded to a multi-layered distal tip 103. FIG. 3 shows a particular example of heat bonding the distal ends of spring-tempered stainless steel dilation elements 110, 120, 130, 140 to a surface of a multi-layered distal tip 103. The dilation elements 110, 120, 130, and 140 are shown as rectangular shaped wires. Referring to FIG. 3, a distal tip 103 is shown comprising a nylon material 193 that is sandwiched between an inner PTFE (polytetraflouroethylene) liner 108 and an outer heat shrink wrap layer 109. The wires 110, 120, 130, and 140 are placed between the distal tip 103 and an outer shrink wrap layer 109. A mandrel may be inserted into the working lumen 150 of the distal tip 103 to prevent the lumen 150 from collapsing during the heat bonding process. The outer diameter of the inner Teflon liner 108 may be etched to create sufficient surface roughness which may provide mechanical adhesion with the nylon distal tip 103 during the heat bonding. The entire multi-layered structure is then heated such that the nylon layer 193, inner Teflon liner 108, and the outer shrink wrap layer 109 partially liquefy at the interface where the layers 103, 108, 109 contact the dilation elements 110, 120, 130, 140. The interface of the dilation elements 110, 120, 130, 140 and layers 103, 109 are melted at a predetermined temperature for a predetermined time. Suitable time, temperature, and pressure parameters are dependent on a variety of factors including the types of materials. The outer shrink tubing 109 reduces in diameter and, in doing so, compresses down over the nylon layer 193 to facilitate the bonding of the layers 101, 108, 109 and the wires 110, 120, 130, 140 to each other. Heat bonding is completed when the materials solidify. The resultant distal tip 103 comprises dilation wires 110, 120, 130, and 140 which are sufficiently rigidly affixed to the distal tip 103 such that they do not rotate or move laterally with respect to each other. FIG. 4 shows an enlarged view of FIG. 3 of one of the dilation elements 110 being heat bonded to the distal tip 103 and sandwiched between the outer shrink tubing 109 and the inner Teflon layer 108.

Although not shown, the proximal end of each of the dilation elements 110, 120, 130, 140 may be heat bonded to a multi-layered catheter 101 in a similar way as shown and described in FIGS. 3 and 4.

Although the above heat bonding example describes an inner Teflon liner 108, an intermediate nylon 193 and outer shrink tubing 109, any polymeric materials may be used. Additionally, although spring-tempered stainless steel is preferred as the dilation element material, any biocompatible material that can be bonded or fastened to a polymeric material may be used. Preferably, the biocompatible metal has sufficient rigidity to access a stenosed region and has sufficient elasticity to enable the dilation elements 110, 120, 130, 140 to return to return to the cage-like orientation upon deflation of the balloon 106.

In an alternative embodiment, referring to FIG. 1, each of the proximal ends of the dilation elements 110, 120, 130, 140 may be bonded within the lumen 107 of the catheter shaft 101 along the distal end of the shaft 101. Similarly, each of the distal ends of the dilation elements 110, 120, 130, 140 may be bonded within the lumen 150 of the distal tip 103. A sufficient amount of the proximal and distal portions of the dilation elements 110, 120, 130, 140 would extend within lumens 107 and 150 to provide a relatively stable heat bond. Each of the dilation elements 110, 120, 130, 140 could be heat bonded at a location at which each of the dilation elements 110, 120, 130, 140 exits the working lumen 107 of the catheter 101 and at a location at which each of the dilation elements 110, 120, 130, 140 enters into a lumen 150 of the distal tip 150 (FIG. 1).

Other means for affixing the proximal and distal ends of the dilation elements 110, 120, 130, 140 to the catheter 101 and the distal tip 103 are contemplated. Although the heat bonding process above was described with the material of the catheter 101 and the distal tip 103 being laminated with multiple layers, the dilation elements 110, 120, 130, 140 may be directly heat bonded to the catheter shaft 101 without using any laminated layers. For example, the dilation elements 110, 120, 130, 140 may be embedded within a homogenous material using heat bonding or insert-molding processes or may be affixed using adhesives. Alternatively, the distal tip 103 may be heated to a liquid state using an insert mold and then the dilation elements 110, 120, 130, 140 may be introduced into the distal tip 103 while the distal tip 103 is molten. The dilation elements 110, 120, 130, 140 may become bonded to the distal tip 103 upon cooling and solidifying. The distal tip 103 may be an injection molded piece with the dilation elements 110, 120, 130, 140 inserted into the mold.

Alternatively, the proximal end of the dilation elements 110, 120, 130, 140 may be held with a fixture or mandrel that is inserted and positioned within the cage-like structure 104 (FIG. 1). The fixture would maintain equal spacing of the dilation elements 110, 120, 130, 140 around the distal end of the catheter 101. Clamping jaws may clamp along the outside of the cage-like structure 104 against the fixture. An adhesive layer may then be applied over the top of the fixture to bond the dilation elements 110, 120, 130, 140 in place. The fixtures may hold the dilation elements 110, 120, 130, 140 to keep them spaced apart and keep their rotational orientation the same. The fixture may be slidable through the cage-like structure 104 similar to a mandrel through the working lumen 107 of the catheter 101 during heat bonding.

In still another embodiment, a collar may be used at the catheter 101 to crimp the proximal end of each of the dilatation elements 110, 120, 130, 140 onto the catheter 101. Another collar may be used at the distal tip 103 to crimp the distal end of each of the dilation elements 110, 120, 130, 140 onto the distal tip. The collars may also comprise radiopaque marker bands for facilitating visualization of the dilation system 100 during a procedure.

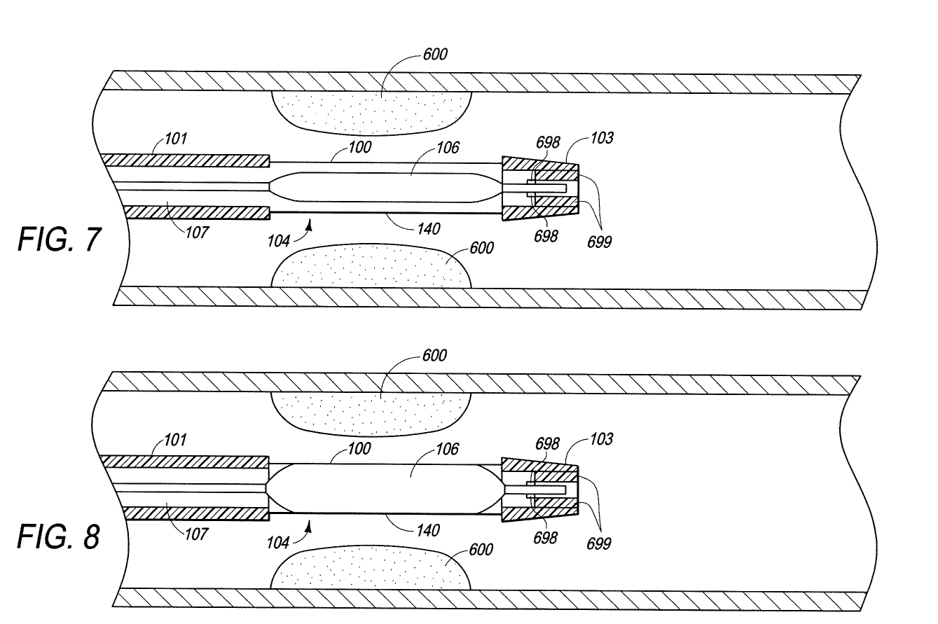

In addition to circular cross-sectional wires, various non-circular cross-sectional shapes may also be used for the dilation elements 110, 120, 130, 140. FIGS. 5A-5E show examples of different cross-sectional shapes of the dilation elements 110, 120, 130, 140 prior to being heat bonded to the intermediate nylon layer 193 between the inner Teflon layer 108 and the outer shrink wrap layer 109. The non-circular shaped dilation elements 110, 120, 130, 140 may help to maintain the position of the dilation elements 110, 120, 130, 140 in their predetermined spaced apart configuration during their radially outward movement as the balloon 106 inflates. FIG. 5A shows rectangular shaped dilation elements 110, 120, 130, 140 disposed between the inner Teflon layer 108 and the outer shrink wrap layer 109. FIG. 5B shows semi-circular/half-round shaped dilation elements 110, 120, 130, 140 disposed between the inner Teflon layer 108 and the outer shrink wrap layer 109. FIG. 5C shows ring shaped fluted dilation elements 110, 120, 130, 140 disposed between the inner Teflon layer 108 and the outer shrink wrap layer 109. FIG. 5D and FIG. 5E show variations of triangular shaped dilation elements 110, 120, 130, 140 disposed between the inner Teflon layer 108 and the outer shrink wrap layer 109. Each of the dilation elements 110, 120, 130, 140 in FIGS. 5A-5E is shown bonded in place to maintain the position of the dilation elements 110, 120, 130, 140 in their predetermined spaced apart configuration during their radially outward movement as the balloon 106 inflates. As described above, the dilation elements 110, 120, 130, 140 are capable of radial flexing inward and outward without undergoing substantial lateral or rotational movement. Although the layers are shown as an inner Teflon layer 108, a middle nylon layer 193, and an outer shrink wrap layer 109, other materials as known to one of ordinary skill in the art may be used as the laminate layers.

Additionally, the different dilation elements 110, 120, 130, 140 may enable the force that is concentrated on a vessel wall to be varied as desired. For instance, the triangular-shaped cross-sectional dilation elements of FIGS. 5D and 5E may in certain applications be preferable over a circular-shaped cross-sectional wire because the triangular-shaped cross-sectional dilation elements may increase the area of the dilation element in contact with the balloon 106 relative to the area of a circular-shaped wire. The triangular-shaped cross-sectional dilation elements may also minimize the area that contacts the stenosed vessel wall relative to the area of a circular-shaped dilation element. Accordingly, a higher stress may be exerted against the stenosed vessel wall by the triangular-shaped dilation elements relative to a circular-shaped dilation element.

The optimal number of dilation elements 110, 120, 130, 140 may vary depending on numerous factors, including the size of the cage-like structure 104, the particular geometry of the stenosed region, the severity of the stenosis, and the type of stenosis to be dilated. Preferably, the number of dilation elements 110, 120, 130, 140 will be sufficient to form a cage-structure 104 with the dilation elements 110, 120, 130, 140 being equidistant from each other. In the example shown in FIG. 1, four rectangular-shaped dilation elements 110, 120, 130, 140 are shown longitudinally aligned with respect to each other and evenly spaced about 90° from each other to form the cage-like structure 104.

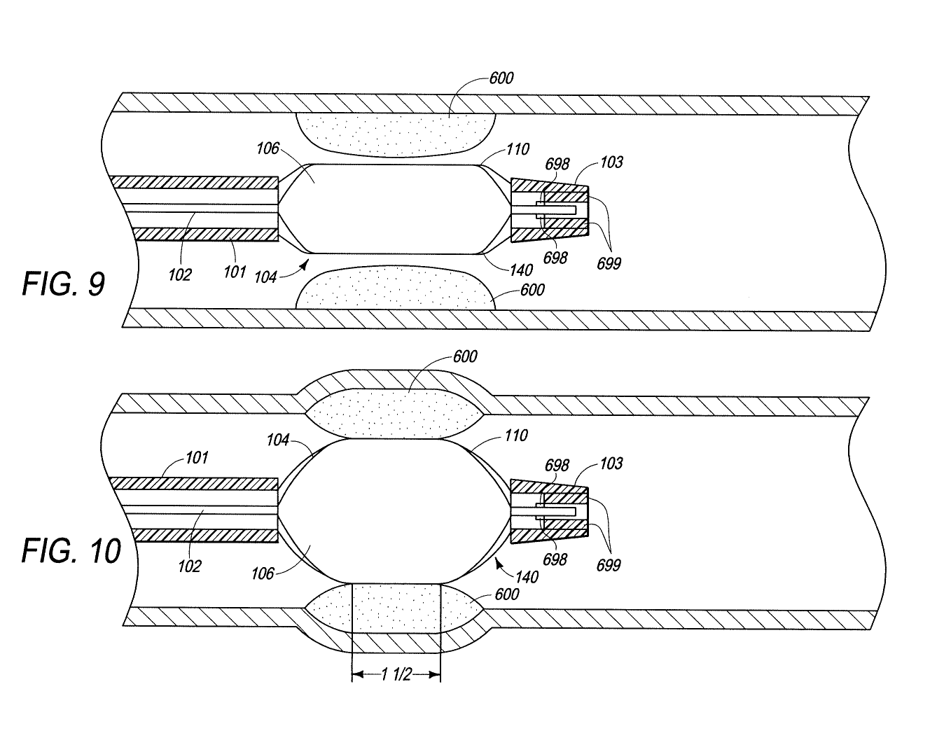

A method of using the dilation system 100 of FIG. 1 may now be described referring to FIGS. 6-10. The balloon catheter 102 is preferably loaded within the cage-like structure 104 of the catheter 101, as shown by the arrow in FIG. 6, prior to being advanced to the stenosed site 600. The loading of the balloon catheter 102 within cage-like structure 104 may also occur after insertion into the body lumen. Radiopaque markers on the catheter 101 and the balloon catheter 102 may be utilized to slide the balloon catheter 102 through the inner passageway 105 of the cage 104 and align the balloon 106 within the cage 104 such that proper placement and fit is achieved between the balloon catheter 102 and the catheter 101, as shown in FIG. 7. Alternatively, a stopper 699 (FIG. 6) may also be affixed on distal tip 103 and a stopper 698 may be affixed on balloon catheter 102 to allow the balloon catheter 102 to properly be positioned and aligned within the cage-like structure 104. In this embodiment, the balloon catheter 102 is inserted into the catheter 101 until stopper 698 abuts against stopper 699, as shown in FIG. 6. A combination of radiopaque markers and stoppers may also be used to ensure proper placement and fit between the balloon catheter 102 and the catheter 101. Yet another embodiment may utilize radiopaque alignment features on the balloon catheter 102, and the catheter 101 to facilitate visual alignment under fluoroscopy. Still another embodiment may utilize reference marks near the proximal ends of both the catheter 101 and the balloon catheter 102 to align the balloon 106 with the cage 104.

After loading of the balloon catheter 102 within the cage-like structure 104, the assembly may be fed over a wire guide which is threaded slightly past the stenosed region 600. Radiopaque markers may be included on the surfaces of the dilation elements 110, 120, 130, 140, the catheter 101, and/or the balloon catheter 102 to facilitate maneuverability to the target stenosed vessel wall 600. Although four dilation elements extend between the catheter 101 and the distal tip 103, it should be noted that only two dilation elements 110 and 140 can be seen in the side views of FIGS. 6-10.

Having positioned the balloon catheter 102-cage like structure 104 to the stenosed region, dilation of the stenosed vessel wall 600 may begin. The balloon 106 may be gradually inflated with saline and/or contrast solution within the cage 104 (FIG. 8). As the balloon 106 inflates such that its circumference begins to increase (FIG. 8), the balloon 106 starts to exert a force against each of the dilation elements 110, 140 thereby causing the dilation elements 110, 140 to bow and be pushed radially outwards.

Because each of the proximal ends of the dilation elements 110, 140 is affixed to the catheter 101 and each of the distal ends of the dilation elements 110, 140 is affixed to the distal tip 103, the ends remain fixated while the unattached portions of the dilation elements 110, 140 radially bow outward along the outer surface of the balloon 106 as shown in FIG. 9 and FIG. 2.

As inflation of the balloon 106 further continues, the dilation elements 110, 140 continue to further radially bow outwards until they contact the stenosed region (FIG. 10). FIG. 10 shows that the balloon 106 may reach its maximum working diameter. Further inflation of the balloon 106 enables the force transmitted through each of the dilation elements 110, 140 to be focused at the regions where each of the dilation elements 110, 140 contacts the stenosed vessel wall 600. Additionally, the dilation elements 110, 140 may distribute the force longitudinally along the length of the balloon 106. This force concentration allows the dilation elements 110, 140 to exert a higher stress at their respective points of contact with the stenosed regions of the vessel wall 600 compared to conventional angioplasty balloons.

The force concentration feature enables dilation of the stenosed vessel wall 600, which may involve cracking and/or fracturing of the calcification rings contained in the blood vessel. After the stenosed vessel wall 600 has been dilated, the balloon 106 may be deflated. The dilation elements 110, 140 may possess spring-like characteristics, which enable the elements 110, 140 to return to their relaxed cage-like configuration 104 as shown in FIG. 7. Upon deflation of the balloon 106, the dilation elements 110, 140 may no longer be in contact with an outer surface of the balloon 106, thereby allowing the balloon catheter 102 to be withdrawn from the cage-like structure 104 of the catheter 101 (FIG. 7).

The dilation mechanism described above may occur at a relatively lower inflation pressure as compared to conventional angioplasty balloons. For example, the balloon catheter 102 of FIG. 1 is adapted to burst a calcification ring surrounding a blood vessel at an inflation pressure ranging between about 4 atm to about 9 atm. The exact inflation pressure is dependent upon numerous factors, including the diameter and geometry of the dilation elements 110, 120, 130, 140 used as well as the size and geometry of the stenosed vessel wall 600. Conventional angioplasty balloons may utilize inflation pressures of about 14 atm to about 15 atm. A lower inflation pressure may be advantageous partly because it reduces the trauma to the stenosed vessel wall 600.

Additionally, the stress exerted by the dilation elements 110, 120, 130, 140 may be predictable and controlled, often requiring a single inflation. Because the dilations are predictable, controlled and often isolated to the stenosed segment of the vessel wall 600, restenosis may be limited to occurring only at the points of contact where the dilation elements 110, 120, 130, 140 exert a stress at their respective points of contact with the stenosed vessel wall 600. Conventional percutaneous transluminal coronary angioplasty (PTCA) procedures typically involve unpredictable points of rupture along the entire circumference of the blood vessel, which often results in more substantial vessel damage to the entire wall. Additionally, multiple inflations may be required to fracture a calcification ring.

The highest degree of cellular proliferation following balloon angioplasty typically occurs in areas with the greatest degree of vessel disruption. Therefore, the ability to dilate a stenotic region in a more controlled and less disruptive manner at a lower pressure, as described with respect to FIGS. 6-10, may potentially minimize the degree of restenosis. Compared to PTCA procedures, the dilation elements 110, 120, 130, 140 may be capable of providing a controlled dilatation in which the injury to the vessel wall is localized to the dilation site only. The balloon catheter 102 may allow relatively lower inflation pressures and a relatively smaller number of inflations to produce significant increases in luminal cross section.

The above described dilation system 100 and method of use thereof possesses several advantages over other types of cutting balloon catheters currently being utilized. The dilation system 100 is relatively inexpensive to manufacture as compared to other cutting balloons. The problem of bonding a wire or other dilation element directly onto a surface of a balloon is a common design challenge encountered in the fabrication of cutting balloons which may lead to relatively expensive design structures. Additionally, the catheter 101 and the distal tip 103 with dilation elements 110, 120, 130, 140 attached thereto may be readily used with a range of different sized balloon catheters. The cage 104 may accommodate a wide range of balloon catheters to dilate a wide array of stenosed vessel walls. This is in contrast to cutting balloons in which a single cutting balloon catheter may only be useful for a certain procedure. As a result, a wide range of different sized cutting balloons may need to be fabricated depending on the stenosed vessel wall intended to be dilated. Furthermore, the balloon catheter 102 may be readily withdrawn from the cage 104, enabling the balloon catheter 102 to be used in other procedures. Because the balloon catheter 102 does not have dilation elements attached to its surface, the balloon catheter 102 is available for a wide range of other applications in which dilation elements may not be needed.

Another advantage of above described dilation system 100 is the ability to interchange balloon catheters within the cage-like structure 104 of catheter 101. The cage-like structure 104 may accommodate a range of different sized balloon catheters. For example, a relatively smaller sized balloon catheter may be replaced with a relatively larger sized balloon catheter during the procedure, if desired. The smaller balloon catheter can be withdrawn through the lumen 107 of the catheter 101 without losing the established pathway from the inlet of the patient's body to the stenosed region 600 so that the procedure can be continued without substantial downtime. The lumen 107 of the catheter 101 also prevents the balloon catheter 102 from abrading against healthy vessel walls when the catheter 102 is withdrawn. Typical angioplasty procedures only have a sheath or shuttle at the entry site of the patient's body rather than along the entire length to the stenosed region. As a result, the insertion and withdrawal of typical multiple balloon catheters, into and from the stenosed region 600 can directly contact the vessel walls and inadvertently traumatize healthy tissue.

Although the balloon catheter 102 and the catheter 101 have been described as preferably delivered together to the target site 600, the balloon catheter 102 and the catheter 101 may be delivered separately (i.e., the balloon 106 may not necessarily reside within the cage-like structure 104 during delivery to the stenosed region 600). For example, if the balloon catheter 102 is being used alone in a conventional angioplasty procedure and it is not until during the procedure that the operator realizes the balloon catheter 102 is not capable of breaking up a hardened stenosis, the cage-like structure 104 may be slidably delivered over the balloon catheter 102 so that dilation elements 110, 120, 130, 140 may crack the calcification ring/hardened stenosis. After cracking the hardened stenosis, the cage-like structure 104 may be retracted sufficiently and conventional angioplasty may resume using balloon 106. Such versatility is not possible using other typical cutting balloons in which the conventional angioplasty balloon catheter would have to be completely withdrawn from the stenosed region 600 and thereafter reintroduced into the stenosed region 600 after the cutting balloon has cracked the calcification ring/hardened stenosis.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A dilation system for dilation of a vessel wall, comprising:
a catheter comprising a distal end and a proximal end;
a distal tip distally spaced apart a predetermined distance from the catheter;
a plurality of dilation elements extending between the catheter and the distal tip, the plurality of dilation elements defining a cage,
a balloon removably and slidably disposable within the cage, the balloon mounted on the distal end of a shaft, wherein at least a length of an outer surface of the balloon comprises a working diameter adapted to dilate the vessel wall, the working diameter of the balloon disposed within the cage, the shaft having an inflation lumen extending therethrough in fluid communication with an interior region of the balloon, the balloon thereby being expandable between a deflated state and an inflated state;
a first stopper element disposed along the distal tip; and
a second stopper element disposed along the shaft of the balloon.

2. The dilation system according to claim 1, wherein each of the plurality of dilation elements is bonded between the distal end of the catheter and a proximal end of the distal tip.

3. The dilation system according to claim 1, wherein each of the plurality of dilation elements is spaced circumferentially apart and is longitudinally aligned with respect to each other.

4. The dilation system according to claim 4, wherein each of the plurality of dilation elements is spaced apart about 90° from each other.

5. The dilation system according to claim 1, wherein the cage is characterized by an inner passageway adapted for the balloon to slide therethrough.

6. The dilation system according to claim 5, wherein the inner passageway comprises a longitudinal length that is at least about equal to a length of the working diameter of the balloon.

7. The dilation system according to claim 1, wherein the plurality of dilation elements are elastically deformable between a cage-like configuration and a radially bowed orientation.

8. The dilation system according to claim 1, wherein each of the proximal ends of the plurality of dilation elements is bonded to the distal end of the catheter.

9. The dilation system according to claim 8, wherein each of the distal ends of the plurality of dilation elements is bonded to a distal end of the distal tip.

10. The dilation system of claim 9, wherein the distal tip comprises laminated layers.

11. The dilation system of claim 10, wherein each of the plurality of dilation elements is bonded to the laminated layers, the laminated layers comprising a middle layer between an outer layer and an inner layer.

12. The dilation system according to claim 1, wherein each of the plurality of dilation elements comprises a width-to-thickness ratio greater than about 1.

13. The dilation system according to claim 1, wherein each of the plurality of dilation elements comprises a non-circular cross section.

14. A dilation system for dilation of a vessel wall, comprising:
a catheter comprising a distal end and a proximal end;
a plurality of wires, each of the plurality of wires comprising a proximal end heat bonded to the distal end of the catheter and a distal end heat bonded to a distal tip, each of the plurality of wires defining a cage, and
a balloon removably and slidably disposed within the cage, the balloon mounted on the distal end of a shaft, wherein at least a length of an outer surface of the balloon comprises a working diameter adapted to dilate the vessel wall, the working diameter of the balloon extending along a length of the balloon, the length of the working diameter being less than the length of the cage, the shaft having an inflation lumen extending therethrough in fluid communication with an interior region of the balloon, the balloon thereby being expandable between a deflated state and an inflated state.

15. The dilation system according to claim 14, wherein each of the catheter and distal tip comprises an inner layer, a middle layer, and an outer layer laminated with respect to each other, the proximal and the distal ends of each of the plurality of wires bonded between the inner, the middle, and the outer layers.

16. The dilation system according to claim 14, wherein each of the plurality of wires comprises a cross-sectional shape that is adapted to bow outwardly in the radial direction.

17. The dilation system according to claim 14, wherein each of the plurality of wires is sufficiently elastic to allow each of the plurality of wires to return from an expanded shape to a collapsed shape.

18. A method of dilating a stenosis in a body vessel, comprising the steps of:
(a) providing a first catheter comprising a cage of dilation elements disposed at a distal end of the first catheter;
(b) providing a second catheter comprising an expandable member
(c) advancing the cage of the first catheter to the target site;
(d) advancing the expandable member of the second catheter to the target site until a first stopper element of the first catheter abuts against a second stopper element of the second catheter; and
(e) expanding the expandable member, wherein each of the plurality of dilation elements expand with the expandable member from a cage-like configuration to a radially expanded configuration toward a stenosed region.

19. The method of claim 18, further comprising the steps of:
(f) deflating the balloon, wherein the dilation elements elastically return to the cage-like configuration;
(g) withdrawing the second catheter from the body vessel; and
(h) withdrawing the first catheter from the body vessel.

20. The method of claim 18, wherein the step (e) of inflating the balloon comprises inflating the balloon to an inflation pressure between about 4 atm to about 9 atm.
